(19) United States
(12) Patent Application Publication
(10) Pub. No.: US 2026/0090570 A1
(43) Pub. Date: Apr. 2, 2026

(54) Cat Food Composition for Treating or Preventing Urolithiasis

(71) Applicant: Blue Buffalo Enterprises, Inc., Wilton, CT (US)

(72) Inventors: Staci L. DeGeer, Wichita, KS (US); Nolan Z. Frantz, Allen, KS (US); Todd Harper, Minneapolis, MN (US); David Kappelman, Olathe, KS (US); Ching-Yen Lin, Bothell, WA (US); Yuka Mitsuhashi, College Grove, TN (US)

(21) Appl. No.: 18/903,721

(22) Filed: Oct. 1, 2024

(51) Int. Cl. A23K 10/30; A23K 10/22; A23K 20/142; A23K 20/24; A23K 20/26; A23K 50/40; A61K 9/00; A61K 31/198; A61K 33/06; A61K 33/42; A61K 35/60; A61K 36/55; A61P 13/12

(52) U.S. Cl. CPC A23K 10/30; A23K 10/22; A23K 20/142; A23K 20/24; A23K 20/26; A23K 50/40; A61K 9/0056; A61K 31/198; A61K 33/06; A61K 33/42; A61K 35/60; A61K 36/55; A61P 13/12

(57) ABSTRACT

A cat food composition comprises ingredients that work to manage urine pH and urine saturation levels to treat or prevent urolithiasis. In some embodiments, a wet cat food composition comprises flaxseed, fish oil, calcium sulfate, methionine and sodium acid pyrophosphate (SAPP). In some embodiments, a dry cat food composition comprises fish oil, methionine, taurine and potassium sulfate. Flaxseed and/or fish oil provide omega-3 fatty acids to minimize urolith formation. Calcium sulfate, potassium sulfate, taurine, methionine and/or SAPP help achieve an ideal urine pH between 6.15 and 6.85. In some embodiments, the cat food composition is configured to establish an average RSS struvite value less than or equal to 1.8, and/or an average RSS oxalate less than or equal to 6.0.

### FIELD OF THE INVENTION

The invention generally pertains to pet food compositions and, more particularly, to cat food compositions formulated to treat or prevent urolithiasis. The invention also pertains to methods of making pet food compositions and, more particularly, to methods of making cat food compositions formulated to treat or prevent urolithiasis.

### BACKGROUND OF THE INVENTION

Feline lower urinary tract disease caused by urinary stones, also called uroliths, is a common ailment in cats. Uroliths cause irritation or blockage and are most often made of struvite (magnesium ammonium phosphate) or calcium oxalate, via a complex biochemical process which includes, e.g., urine supersaturation and crystal growth. A therapeutic or preventative diet for managing mineral levels and other factors affecting urolith formation is needed for cat owners and veterinarians to manage the urinary health of their cats.

### SUMMARY OF THE INVENTION

A cat food composition in accordance with the present invention comprises ingredients that work to manage urine pH and urine saturation levels. In some embodiments, the cat food composition is a wet cat food composition comprising flaxseed, fish oil, calcium sulfate, methionine and sodium acid pyrophosphate (SAPP). In some embodiments, the cat food composition is a dry cat food composition comprising fish oil, methionine, taurine and potassium sulfate.

Flaxseed (e.g., flaxseed meal) and/or fish oil are included in the cat food composition of the present invention to provide omega-3 fatty acids to minimize urolith formation. In some embodiments, flaxseed (e.g., flaxseed meal) is present in the cat food composition in an amount of about 0.1% to about 10% (e.g., about 0.5% to about 5% or about 0.25% to about 2.5%). In some embodiments, fish oil (e.g., Menhaden fish oil) is present in the cat food composition in an amount of about 0.1% to about 10% (e.g., about 0.5% to about 5% or about 0.25% to about 2.5%).

Calcium sulfate, potassium sulfate, taurine, methionine (e.g., DL-methionine) and/or SAPP are included in the cat food composition of the present invention to help achieve an ideal urine pH (e.g., a pH between 6.15 and 6.85). In some embodiments, the cat food composition of the present invention includes calcium sulfate in an amount of about 0.04% to about 4% (e.g., about 0.2% to about 2% or about 0.1% to about 1%), potassium sulfate in an amount of about 0.1% to about 0.6%, taurine in an amount of about 0.1% to about 1.0%, methionine in an amount of about 0.002% to about 0.2% (e.g., about 0.01% to about 0.1% or about 0.005% to about 0.05%), and/or SAPP in an amount of about 0.015% to about 1.5% (e.g., about 0.075% to about 0.75% or about 0.045% to about 0.45%).

In some embodiments, the cat food composition is configured to establish a urine pH between 6.15 and 6.85 (e.g., between 6.15 and 6.56 or between 6.2 and 6.4 for the wet cat food embodiments, between 6.31 and 6.85 for the dry cat food embodiments). This pH range is ideal to prevent the formation of struvite and/or calcium oxalate crystals. In some embodiments, the cat food composition has a base excess within the range of about-80 to about-100 (e.g., for the wet cat food embodiments) or about-50 to about-200 (e.g., for the dry cat food embodiments). In some embodiments, the cat food composition has a calcium to phosphorous ratio in the range of 1 to 1.5. In some embodiments, the cat food composition is configured to establish an average RSS struvite value less than or equal to 1.8, and/or an average RSS oxalate less than or equal to 6.0.

A method of making a cat food composition for treating or preventing urolithiasis in accordance with the present invention comprises combining flaxseed, fish oil, calcium sulfate, methionine and sodium acid pyrophosphate or comprises combining fish oil, methionine, taurine and potassium sulfate. In some embodiments, combining includes combining flaxseed in an amount of about 0.1% to about 10% (e.g., about 0.5% to about 5% or about 0.25% to about 2.5%) by weight of the cat food composition, fish oil in an amount of about 0.1% to about 10% (e.g., about 0.5% to about 5% or about 0.25% to about 2.5%) by weight of the cat food composition, calcium sulfate in an amount of about 0.04% to about 4% (e.g., about 0.2% to about 2% or about 0.1% to about 1%) by weight of the cat food composition, methionine in an amount of about 0.002% to about 0.2% (e.g., about 0.01% to about 0.1% or about 0.005% to about 0.05%) by weight of the cat food composition and sodium acid pyrophosphate in an amount of about 0.015% to about 1.5% (e.g., about 0.075% to about 0.75% or about 0.045% to about 0.45%) by weight of the cat food composition. In some embodiments, combining includes combining fish oil in an amount of about 0.1% to about 10% (e.g., about 0.5% to about 5% or about 0.25% to about 2.5%) by weight of the cat food composition, methionine in an amount of about 0.002% to about 0.2% (e.g., about 0.01% to about 0.1% or about 0.005% to about 0.05%) by weight of the cat food composition, taurine in an amount of about 0.1% to about 0.6% by weight of the cat food composition and potassium sulfate in an amount of about 0.1% to about 1.0% by weight of the cat food composition.

### DETAILED DESCRIPTION OF EMBODIMENTS

Initially, it should be noted that the embodiments of the present invention described below are not intended to be exhaustive or limit the invention to the precise forms disclosed in the following detailed description. Rather, a purpose of the embodiments chosen and described is so that the appreciation and understanding by others skilled in the art of the principles and practices of the present invention can be facilitated.

Throughout the present description, unless otherwise specified, the concentrations expressed as percentages always refer to the weight/weight (w/w) percentage, i.e., grams of a given component per 100 g of composition, i.e., wt % or percent by weight. The term “about” refers to a deviation of up to plus/minus 10%, preferably plus/minus 5%.

The level of urinary stone formation in a cat's urinary system is dependent upon many factors including but not limited to urine mineral content, urine pH and the presence of inhibitors and promotors (e.g., magnesium cations) of stone formation. For example, struvite is more likely to form as urine pH increases while calcium oxalate is more likely to form as urine pH decreases. The present inventors have developed a cat food composition with precise mineral levels and added polyunsaturated fatty acid levels to manage a cat's urine pH and urine saturation levels so as to control or even reverse the formation of struvite and calcium oxalate crystals/stones which are a common cause of feline lower urinary tract disease. The cat food composition may meet the requirements for the Association of American Feed Control Officials (AAFCO) “natural” designation (e.g., not containing any additives or processing aids that are chemically synthetic except in amounts as might occur in good manufacturing practices) and contains no added corn, wheat, or soybean ingredients and no artificial flavors or colorants. The cat food composition may be considered a natural food for cats with added vitamins and minerals. The cat food may also not contain any chicken by-product meal.

In some embodiments, the cat food composition is a wet cat food composition comprising flaxseed, fish oil, calcium sulfate, methionine and sodium acid pyrophosphate. The wet cat food composition of the present invention can be a semi-moist cat food composition (i.e., having a total moisture content of from 10% to 50%, by weight), and/or a moist cat food composition (i.e., having a total moisture content of greater than 50%, by weight, e.g., about 76% moisture). In some embodiments, the cat food composition is a dry cat food composition comprising fish oil, methionine, taurine and potassium sulfate. The dry cat food composition of the present invention can be a kibble cat food composition (i.e., having a total moisture content of from 5% to 12%, by weight). The cat food composition can further include ingredients such as water; animal protein such as beef, chicken or fish; farinaceous matter such as brown rice flour or potato; plant protein such as pea protein; chicken broth; guar gum; taurine; potassium chloride; potassium sulfate; choline chloride; vitamin E supplement; salt; and/or fruits such as blueberries or cranberries.

Flaxseed (e.g., flaxseed meal) and/or fish oil are included in the cat food composition of the present invention to provide omega-3 fatty acids such as eicosapentaenoic acid (EPA) and docosahexaenoic acid (DHA). Polyunsaturated fatty acids such as those in flaxseed and fish oil are used to minimize urolith formation. In some embodiments, flaxseed (e.g., flaxseed meal) is present in the cat food composition in an amount of about 0.1% to about 10% (e.g., about 0.5% to about 5% or about 0.25% to about 2.5%). In some embodiments, fish oil (e.g., Menhaden fish oil) is present in the cat food composition in an amount of about 0.1% to about 10% (e.g., about 0.5% to about 5% or about 0.25% to about 2.5%).

Calcium sulfate, potassium sulfate, taurine, methionine (e.g., DL-methionine) and/or SAPP are included in the cat food composition of the present invention to help achieve an ideal urine pH (e.g., a pH between 6.15 and 6.85). In particular, calcium sulfate, potassium sulfate, taurine, or methionine are included as sources of sulfur to help achieve an ideal urine pH while sodium acid pyrophosphate is added as a mineral source of phosphorous to help achieve an ideal urine pH. In some embodiments, the cat food composition of the present invention includes calcium sulfate in an amount of about 0.04% to about 4% (e.g., about 0.2% to about 2% or about 0.1% to about 1%), potassium sulfate in an amount of about 0.1% to about 0.6%, taurine in an amount of about 0.1% to about 1.0%, methionine in an of about 0.002% to about 0.2% (e.g., about 0.01% to about 0.1% or about 0.005% to about 0.05%), and/or SAPP in an amount of about 0.015% to about 1.5% (e.g., about 0.075% to about 0.75% or about 0.045% to about 0.45%).

Controlled levels of added magnesium help prevent the formation of struvite crystals while controlled levels of added calcium help prevent the formation of calcium oxalate crystals. In some embodiments, the cat food composition does not include an added magnesium supplement ingredient (i.e., does not include magnesium outside of magnesium naturally occurring in meat or plant sources ingredients in the cat food composition) so as not to promote stone formation.

In some embodiments, the cat food composition is configured to establish a urine pH between 6.15 and 6.85 (e.g., between 6.15 and 6.56 or between 6.2 and 6.4 for the wet cat food embodiments, between 6.31 and 6.85 for the dry cat food embodiments). This pH range is ideal to prevent the formation of struvite and/or calcium oxalate crystals. In some embodiments, the cat food composition has a base excess within the range of about-80 to about-100 (e.g., for the wet cat food embodiments) or about-50 to about-200 (e.g., for the dry cat food embodiments). The “base excess” (BE) of the cat food composition is an indication of the balance of its component cations and anions and can correlate with the cat's urine pH. BE can be calculated from the compounds in the cat food composition as detailed in Jeremias et al. “Predictive formulas for food base excess and urine pH estimations of cats.” Animal feed science and technology 182, no. 1-4 (2013): 82-92, which is incorporated herein by reference.

Another important aspect of the cat food composition is its calcium (Ca) to phosphorous (P) ratio. In some embodiments, the cat food composition has a calcium to phosphorous ratio in the range of 1 to 1.5 (i.e., Ca:P of from 1:1 to 1.5:1).

Relative supersaturation (RSS) is a measurement used to assess the risk of crystal formation in urine and is based upon urine pH and urinary concentration of select solutes (e.g., struvite or calcium oxalate). An RSS value of less than 1.0 for struvite and less than 5.0 for calcium oxalate is known to result in crystal dissolution. In some embodiments, the cat food composition is configured to establish an average RSS struvite value less than or equal to 1.8, and/or an average RSS oxalate less than or equal to 6.0. See Example 2 below.

The present invention is also directed to a method of making a cat food composition for treating or preventing urolithiasis. The method comprises combining flaxseed, fish oil, calcium sulfate, methionine and sodium acid pyrophosphate (e.g., to form a wet cat food composition) or comprises combining fish oil, methionine, taurine and potassium sulfate (e.g., to form a dry cat food composition). In some embodiments, combining includes combining flaxseed in an amount of about 0.1% to about 10% (e.g., about 0.5% to about 5% or about 0.25% to about 2.5%) by weight of the cat food composition, fish oil in an amount of about 0.1% to about 10% (e.g., about 0.5% to about 5% or about 0.25% to about 2.5%) by weight of the cat food composition, calcium sulfate in an amount of about 0.04% to about 4% (e.g., about 0.2% to about 2% or about 0.1% to about 1%) by weight of the cat food composition, methionine in an amount of about 0.002% to about 0.2% (e.g., about 0.01% to about 0.1% or about 0.005% to about 0.05%) by weight of the cat food composition and sodium acid pyrophosphate in an amount of about 0.015% to about 1.5% (e.g., about 0.075% to about 0.75% or about 0.045% to about 0.45%) by weight of the cat food composition. In some embodiments, combining includes combining fish oil in an amount of about 0.1% to about 10% (e.g., about 0.5% to about 5% or about 0.25% to about 2.5%) by weight of the cat food composition, methionine in an amount of about 0.002% to about 0.2% (e.g., about 0.01% to about 0.1% or about 0.005% to about 0.05%) by weight of the cat food composition, taurine in an amount of about 0.1% to about 0.6% by weight of the cat food composition and potassium sulfate in an amount of about 0.1% to about 1.0% by weight of the cat food composition.

In some embodiments, the method further comprises making a cat food composition configured to establish a urine pH between 6.15 and 6.85 (e.g., between 6.15 and 6.56 or between 6.2 and 6.4 for the wet cat food embodiments, between 6.31 and 6.85 for the dry cat food embodiments). In some embodiments, the method further comprises making a cat food composition having a base excess within the range of about-80 to about-100 (e.g., for the wet cat food embodiments) or about-50 to about-200 (e.g., for the dry cat food embodiments). In some embodiments, the method further comprises making a cat food composition having a calcium to phosphorous ratio in the range of 1 to 1.5. In some embodiments, the method further comprises making a cat food composition configured to establish an average RSS struvite value less than or equal to 1.8, and/or an average RSS oxalate less than or equal to 6.0.

### EXAMPLES

Example 1-Product Formulation. Table 1 illustrates select ingredients and associated weight percentages for exemplary cat food compositions in accordance with the present invention. Table 2 illustrates select nutrient content of the exemplary cat food compositions. The Concept5 Formulation System from Creative Formulation Concepts (CFC) was used to compute nutrient values, ratios and base excess. The exemplary wet cat food composition was computed to have a base excess of −100.47 and a Ca to P ratio of 1.07.

TABLE 1

| | Exemplary Wet Cat Food | Exemplary Dry Cat Food |
|---|---|---|
| | Percentage of product | Percentage of product |
| Ingredient | by weight (%) | by weight (%) |
| Water | 10-50 | 5-12 |
| Flaxseed meal | 0.5-5.0 | NA |
| Menhaden fish oil | 0.25-2.5 | 0.1-2.0 |
| Calcium sulfate | 0.2-2.0 | NA |
| SAPP | 0.01-0.1 | NA |
| Methionine | 0.075-0.75 | 0.1-0.8 |
| Taurine | NA | 0.1-0.6 |
| Potassium Sulfate | NA | 0.1-1.0 |

TABLE 2

| | Exemplary Wet Cat Food | Exemplary Dry Cat Food |
|---|---|---|
| | Percentage of product | Percentage of product |
| Nutrient | by weight (%) | by weight (%) |
| Calcium | 0.30-0.66 | 0.60-1.00 |
| Phosphorous | 0.30-0.51 | 0.50-0.80 |
| Sodium | 0.10-0.30 | 0.40-0.80 |
| Magnesium | 0.02-0.04 | 0.04-0.10 |
| Sulfur | 0.10-0.30 | 0.45-0.80 |
| Potassium | 0.20-0.40 | 0.55-0.90 |

Example 2-Testing. A 42-day Relative Supersaturation (RSS) feeding test was performed on a panel of cats to validate the performance of the above-mentioned exemplary cat food compositions. A test diet was offered twice a day. A urine sample was collected for each of the cats on days 10, 20, 30, and 40 of the feeding test and tested for pH, struvite RSS and calcium oxalate RSS. The target RSS struvite value was an average of less than or equal to 1.8 and an upper 95% confidence level equal to or less than 2.5. The target RSS calcium oxalate value was an average of less than or equal to 6.0 with an upper 95% confidence level of less than or equal to 12.0. The average urine pH over all days tested was 6.32, with an average of 0.26 for struvite relative supersaturation and 1.28 for calcium oxalate relative supersaturation.

1. A cat food composition for treating or preventing urolithiasis, the composition comprising:
flaxseed;
fish oil;
calcium sulfate;
methionine; and
sodium acid pyrophosphate.

2. The cat food composition of claim 1, wherein the cat food composition is a wet cat food composition.

3. The cat food composition of claim 1, wherein flaxseed is present in an amount of about 0.1% to about 10% by weight of the cat food composition, fish oil is present in an amount of about 0.1% to about 10% by weight of the cat food composition, calcium sulfate is present in an amount of about 0.04% to about 4% by weight of the cat food composition, methionine is present in an amount of about 0.002% to about 0.2% by weight of the cat food composition and sodium acid pyrophosphate is present in an amount of about 0.015% to about 1.5% by weight of the cat food composition.

4. The cat food composition of claim 1, wherein the cat food composition is configured to establish a urine pH between 6.15 and 6.56.

5. The cat food composition of claim 1, wherein the cat food composition has a base excess within the range of about-80 to about-100.

6. The cat food composition of claim 1, wherein the cat food composition has a calcium to phosphorous ratio in the range of 1 to 1.5.

7. The cat food composition of claim 1, wherein the cat food composition is configured to establish an average RSS struvite value less than or equal to 1.8, and/or an average RSS oxalate less than or equal to 6.0.

8. A cat food composition for treating or preventing urolithiasis, the composition comprising:
fish oil;
potassium sulfate;
methionine; and
taurine.

9. The cat food composition of claim 8, wherein the cat food composition is a dry cat food composition.

10. The cat food composition of claim 8, wherein fish oil is present in an amount of about 0.1% to about 1.0% by weight of the cat food composition, potassium sulfate is present in an amount of about 0.1% to about 0.6% by weight of the cat food composition, methionine is present in an amount of about 0.002% to about 0.2% by weight of the cat food composition and taurine is present in an amount of about 0.1% to about 1.0% by weight of the cat food composition.

11. The cat food composition of claim 8, wherein the cat food composition is configured to establish a urine pH between 6.31 and 6.85.

12. The cat food composition of claim 8, wherein the cat food composition has a base excess within the range of about-50 to about-200.

13. The cat food composition of claim 8, wherein the cat food composition has a calcium to phosphorous ratio in the range of 1 to 1.5.

14. The cat food composition of claim 8, wherein the cat food composition is configured to establish an average RSS struvite value less than or equal to 1.8, and/or an average RSS oxalate less than or equal to 6.0.

15. A method of making a cat food composition for treating or preventing urolithiasis, the method comprising combining flaxseed,
fish oil,
calcium sulfate,
methionine and
sodium acid pyrophosphate;
or combining
fish oil,
potassium sulfate,
methionine and
taurine.

16. The method of claim 15, wherein combining includes combining flaxseed in an amount of about 0.1% to about 10% by weight of the cat food composition, fish oil in an amount of about 0.1% to about 10% by weight of the cat food composition, calcium sulfate in an amount of about 0.04% to about 4% by weight of the cat food composition, methionine in an amount of about 0.002% to about 0.2% by weight of the cat food composition and sodium acid pyrophosphate in an amount of about 0.015% to about 1.5% by weight of the cat food composition.

17. The method of claim 15, wherein combining includes combining fish oil in an amount of about 0.1% to about 10% by weight of the cat food composition, methionine in an amount of about 0.002% to about 0.2% by weight of the cat food composition, taurine in an amount of about 0.1% to about 0.6% by weight of the cat food composition and potassium sulfate in an amount of about 0.1% to about 1.0% by weight of the cat food composition.
